(19) United States
(12) Patent Application Publication
(10) Pub. No.: US 2025/0276143 A1
(43) Pub. Date: Sep. 4, 2025

(54) A DILATING AND STENTING ORAL OR NASOPHARYNGEAL AIRWAY

(71) Applicant: Board of Regents, The University of Texas System, Austin, TX (US)

(72) Inventors: Steven G. Venticinque, San Antonio, TX (US); Justin Rice, Denver, CO (US); Travis Chastain, San Antonio, TX (US); Jay Johannigman, San Antonio, TX (US)

(21) Appl. No.: 18/860,871

(22) PCT Filed: Apr. 28, 2023
(86) PCT No.: PCT/US2023/020309

§ 371 Date: Oct. 28, 2024

Related U.S. Application Data
(60) Provisional application No. 63/336,956, filed on Apr. 29, 2022.

(51) Int. Cl. A61M 16/04

(52) U.S. Cl. CPC A61M 16/0461; A61M 16/0475

(57) ABSTRACT

Aspects of the application are directed to a nasopharyngeal airway (NPA) or an oral airway (OA) and methods of deploying same, the NPA or OA comprising an elongated body forming a lumen, the elongated body having a proximal end to be positioned outside of a patient during use and a distal end to be inserted into a patient's airway during use, the elongated body having a distal expandable portion configured to be positioned in the pharyngeal segment of a patient airway which when self-expanded provides airflow patency within the airway in which the NPA or OA is inserted due to stenting open of the soft tissues, the central lumen of the NPA or OA, and through fenestrations, transverse openings, or fins.

### RELATED APPLICATIONS

This application claims priority to U.S. Provisional Patent Application Ser. No. 63/336,956 filed Apr. 29, 2022 which is incorporated herein by reference in its entirety.

### STATEMENT REGARDING FEDERALLY FUNDED RESEARCH

None.

### BACKGROUND

Health care providers utilize nasopharyngeal airway (NPA) devices to prevent or treat upper airway obstruction in patients who are breathing spontaneously or in patients who require assisted breathing via positive pressure mask ventilation. A typical nasopharyngeal airway device comprises an elongated tube having openings located at the top end and at the bottom end so that air can pass through (FIG. 1). The NPA or nasal trumpet (sometimes called a nasal “trumpet” because of its flared proximal end), is designed to be inserted through the nose and into the upper airway of an unconscious or semiconscious patient to relieve breathing obstruction caused by soft tissue collapse from the soft palate and structures within the oropharynx (FIG. 2). NPAs are constructed out of soft rubber or silicone-like material allowing them to be stiff enough to insert without buckling but soft enough to prevent serious trauma to soft tissues. The devices are inserted into one of the nasal passageways of the patient, thereby providing a conduit through which the patient can easily breathe. For example, NPAs are commonly used in patients who remain sedated from anesthesia following an operation and who have difficulty breathing due to anesthesia-induced collapse of their upper airway soft tissues. Another scenario might involve relieving airway obstruction in a patient who is comatose due to severe illness or critical injury who is attempting to breathe spontaneously or is receiving ineffective mask-assisted positive pressure ventilation.

The NPA is one of the most basic lifesaving airway management tools. It is a 140-year-old technology that has remained largely unchanged since its inception. Due to its simplicity of operation and ease of insertion the NPA can be utilized by medical personnel with minimal training and experience. Because of this, NPAs are considered an essential supply item within hospital and prehospital airway kits and trolleys. However, despite the NPA's long history and wide distribution it is not universally effective in relieving airway upper obstruction. Furthermore, the NPA appears to be underutilized resulting in potential unnecessary airway interventions that many medical personnel are ill-suited to perform (e.g., endotracheal intubation and emergency cricothyroidotomy).

Upper airway patency depends upon an appropriate balance between airway structural components, the dilating force of the pharyngeal muscles, and the collapsing influence of negative intraluminal pressure in the airway caused by inspiratory airflow. The neuromuscular activity of upper airway stabilizing muscles are greatly diminished during sleep, sedation, and altered states of consciousness such as the lethargy and coma that can be associated with shock, brain injury, and severe metabolic disturbances. These states are associated with decreased pharyngeal caliber, increased upper airway resistance, and ultimately closure within the collapsible segment of the upper airway (FIG. 3A). During normal conditions, the pressure within the pharynx (P_{PHARYNX}) exceeds the pressure exerted by the surrounding tissues (P_{TISSUE}) and the airway remains patent (FIG. 3B). As the dilating force of the pharyngeal muscles decreases with diminished consciousness P_{TISSUE} increases and the airway begins to narrow. This in turn leads to increased air flow velocity, which decreases P_{PHARYNX} (FIG. 3C). The moment P_{PHARYNX} pressure drops below surrounding P_{TISSUE}, the airway collapses, and airflow stops (FIG. 3 Panel D).

The principal points of obstruction within the collapsible segment of the pharynx occur at the levels of the soft palate, tongue base, oropharynx, and the epiglottis (Refer to FIG. 2). A properly sized NPA will pass below the soft palate with the tip residing in the oropharynx approximately 1 cm above the epiglottis. This position will provide relief of obstruction at the soft palate level if the distal tip or the lumen of the NPA does not become obstructed with pharyngal soft tissue or secretions (FIG. 4A). However, the effectiveness of the NPA in reliably relieving obstruction at the level of oropharynx, tongue base and epiglottis is not fully understood. In fact, despite the presence of an NPA, airway obstruction can occur in up to 42% of heavily sedated patients.

### SUMMARY

The inventors have designed a novel airway device that is founded upon a contemporary understanding of the complexity of multi-level upper airway collapse. The technology addresses these anatomic challenges by gently dilating and stenting open the upper airway at multiple levels including the soft palate, oropharynx, tongue base, and the region of the epiglottis (FIG. 4B and FIG. 5). The term stenting open refers to the positioning of a support structure inserted into the lumen of a passageway to keep the passageway open. This results in an increase in pharyngeal caliber along multiple pharyngeal segments and more reliable upper airway patency. Furthermore, the airway opening action of the dilated section of the airway device is transmitted distally and proximally resulting in a clinical effect beyond the limits of the dilating segment. Since the collapsible forces of the upper airway are negligible, the device can be soft resulting in atraumatic insertion and removal. The device also allows for alternate air flow pathways, as opposed to the single-lumen traditional NPA, through numerous fenestrations within the dilatation segment, the body, or the dilated segment and the body. The term “fenestrated” is defined as the quality of possessing macroscopic perforations or holes in an otherwise solid or hollow component. “Fenestrations” refers to the holes or perforations. This can facilitate oral ventilation and the oral evacuation of secretions from the airway device lumen. The airway device described herein is designed to eliminate upper airway obstruction during both spontaneous breathing and mask-assisted positive pressure ventilation.

Certain embodiments described herein enhance the effectiveness of the traditional airways, nasopharyngeal airway (NPA) and oral airway (OA). The airway devices described herein is founded upon the most recent anatomic knowledge of dynamic upper airway collapse, that utilizes modern material technology, and is significantly more effective in relieving airway obstruction.

Certain embodiments are directed to a nasopharyngeal airway (NPA) comprising an elongated body forming a lumen, the elongated body having a proximal end to be positioned outside of a patient during use and a distal end to be inserted into a patient airway during use. The elongated body having a distal expandable/dilating portion or segment configured to be positioned in the pharyngeal segment of a patient airway which when expanded or self-expanded provides airflow patency within the airway in which the NPA is inserted due to stenting open of the soft tissues. The airflow patency is also provided or enhanced using the central lumen of the NPA, and through fenestrations, transverse openings, and/or fins. In certain aspects the expandable portion includes foldable fins and transverse openings or fenestrations forming a fluid path between the inner lumen and exterior of the NPA. The expandable portion can include a collapsible and/or self-expanding lattice structure with fenestrations forming a fluid path between the inner lumen and exterior of the NPA. The NPA can be 8, 9, 10, 11, 12, 13, 14, 15, 16, 17, 18, 19, 20, 21, 22, 23, 24, 25, 26, 27, 28, 29 to 30 cm in length (including all values or ranges there between), with an outer diameter ranging from 0.4, 1, 2, 3, 4, to 5 cm (including all values or ranges there between), and an inner diameter of 0.2, 0.3, 0.4, 0.5, 0.6, 0.7, 0.8, 0.9, 1, 2, to 3 cm. In certain aspects the NPA can be 6, 8, 10, or 12 to 20, 22, or 25 cm in length. In other aspects the outer diameter can be 0.4 to 2.5 cm. In certain aspects the inner diameter is 0.2 to 2.3 cm. The distal expandable portion is 2, 3, 4, 5, 6, 7, 8, 9, to 10 cm (including all values or ranges there between) in length and having an expanded outer diameter of 1, 1.5, 2, 2.5, 3, 3.5, 4, 4.5, 5, 5.5, to 6 cm (including all values or ranges there between). The distal expandable portion can be positioned 0.2, 0.3, 0.4, 0.5, 1, 2, 3, 4, to 5 cm from the distal tip or the beginning of a distal beveled tip of the body. In certain aspects NPA may have a general curvature or contour to aid in the insertion of the device. The radius of curvature of the NPA or any of the disclosed embodiments may be between a straight device and approximately 10, 20, 40, 50, 60, 70, 80, 90, 100, 125, 150, 175, 200, 225, 250, 275, 300, 350, 400, 450, 500 millimeters (mm), or between 60 mm and 500 mm, between approximately 100 mm and 500 mm. In certain aspects a 2, 3, 4, 5, 6, 7, 8, 9, to 10 cm portion of the device is curved while the remaining portions are straight. The term “radius of curvature” refers to the radius of the circle in a horizontal plane that best fits the curve. The NPA can be made of a polymer material. Polymer material refers to compounds composed of linked repeating bonds and include thermosetting and thermoplastic polymers-such as thermoplastic plastics selected from PE, PP, PVC, PS, ABS, PEEK, PES, PASU, PPS, XLPE, m-PP and PB-1; thermosetting plastics selected from phenolics, epoxy, amino, unsaturated polyester, furane, polysiloxane, and PDAP plastics; rubbers selected from styrene butadiene rubber, butadiene rubber, isoprene rubber, ethylene propylene rubber and neoprene rubber; and special rubbers selected from nitrile rubber, chlorinated rubber, fluororubber, epichlorohydrin rubber, silicone rubber, urethane rubber, polysulfide rubber, and acrylate rubber. The term “rubber” as used herein, is intended to include all elastic materials, such as natural rubber, synthetic rubber, and the like. The distal most 1 to 2 cm can be a beveled tip. The NPA can be coupled with a central introducer to facilitate insertion of the NPA into a patient by providing axial rigidity, collapse a self-expanding lattice structure, and/or allow the lattice structure to expand as the introducer is removed. The central introducer can be temporarily secured in place by a locking mechanism on the proximal end of the NPA. The NPA can include a removable mesh, filament, cord, or string to hold or restrain the self-expanding lattice structure closed to facilitate insertion, and then allow the lattice structure to expand or self-expand as the introducer, removable mesh, filament, cord, or string is removed.

Certain embodiments are directed to a method for inserting the NPA, the method including the steps of advancing a distal end of an NPA through a nostril opening into a nasal passageway of a patient and then caudally into the pharynx; the NPA (as described above) having a distal end that will collapse during insertion or be held collapsed by an introducer, mesh, filament, cord, or string, and then deploying the NPA in position by self-expanding in the pharynx naturally or after releasing a restraint deploying stenting fins or a lattice structure increasing the luminal diameter of the airway and providing alternate paths for airflow.

Certain embodiments are directed to an oral inserted airway (OA) comprising an elongated body or body member forming a lumen, the elongated body having a proximal end to be positioned outside of a patient during use and a distal end to be inserted into a patient oral airway during use. The elongated body having a distal expandable portion or segment configured to be positioned in the oral airway of a patient or subject which when expanded or self-expanded provides airflow patency within the oral airway in which the OA is inserted due to stenting open of the soft tissues or preventing collapse of soft tissues. The airflow patency is also provided or enhanced using the central lumen of the OA, and through fenestrations, transverse openings, and/or fins. In certain aspects the expandable portion includes foldable fins and transverse openings or fenestrations forming a fluid path between the inner lumen and exterior of the OA. The expandable portion can include a collapsible and/or self-expanding lattice structure with fenestrations forming a fluid path between the inner lumen and exterior of the OA. The OA can be 3, 4, 5, 6, 7, 8, 9, 10, 11, 12, 13, 14, to 15 cm in length (including all values and ranges there between), with an outer diameter ranging from 0.5, 1, 2, 3, 4, 5 cm, and an inner diameter of 0.1, 0.2, 0.3, 0.4, 0.5, 0.6, 0.7, 0.8, 0.9, 1, 1.5, 2, 2.5, 3, 3.5, to 4 cm (including all values and ranges there between). The distal expandable portion is 0.1, 1, 2, 3, 4, 5, 6, 7, 8, 9, to 10 cm in length (including all values and ranges there between) and having an expanded outer diameter of 1, 1.5, 2, 2.5, 3, 3.5, 4, 4.5, 5, 5.5, to 6 cm (including all values and ranges there between). In certain aspects the OA may have a general curvature or contour to aid in the insertion of the device. The radius of curvature of the OA or any of the disclosed embodiments may be between a straight device and approximately 10, 20, 40, 50, 60, 70, 80, 90, 100, 125, 150, 175, 200, 225, 250, 275, 300, 350, 400, 450, 500 millimeters (mm), or between 60 mm and 500 mm, between approximately 100 mm and 500 mm. In certain aspects a 2, 3, 4, 5, 6, 7, 8, 9, to 10 cm portion of the device is curved while the remaining portions are straight with no curvature. The term “radius of curvature” refers to the radius of the circle in a horizontal plane that best fits the curve. The distal expandable portion can be positioned 0.1, 0.25, 0.5, 1, 2, 3, to 4 cm (including all values and ranges there between) from the distal tip or the beginning of a distal beveled tip of the body. The OA can be made of a polymer material. The distal most 0.2 to 3 cm (including all values and ranges there between) can be a beveled tip. The OA can be coupled with a central introducer to facilitate insertion of the OA into a patient which can, but not necessarily provide one or more of axial rigidity, collapse a self-expanding lattice structure, and/or allow the lattice structure to expand as the introducer is removed. The central introducer can be temporarily secured in place by a locking mechanism on the proximal end of the OA. The OA can include a removable mesh, filament, cord, or string to hold or restrain the self-expanding lattice structure closed to facilitate insertion, and then allow the lattice structure to self-expand as the removable mesh, filament, cord, or string is removed. The OA can be a curved hollow, tubular longitudinally extending body. In certain aspects the curvature of the body is between 0° 10, 20, 30, 40, 50, 60, 70, 80, 90, 100, 110, 120, 130, to 140°. The body can include a flanged proximal end portion for location at the mouth of the patient. In certain aspects the flanged proximal end portion is configured as a bite block or is configured to connect to a bite block. The body member further includes a distal end portion for insertion into the mouth and pharynx of the patient. The central lumen of the OA can be but is not limited to a round or an oval shape.

The term “airway device” includes nasopharyngeal airway devices (NPAs) and oral airway devices (OAs).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. An illustration of a prior art Nasopharyngeal Airway (NPA).

FIG. 2. An illustration of a prior art NPA in place with upper airway anatomy.

FIG. 3A-3D model illustrating the physiology of upper airway collapse. (A) The upper airway is composed of a collapsible central segment (the pharynx) that is surrounded by rigid, non-collapsible segments. (B) Due to a lack of rigid supporting airway structures, the oropharyngeal segment of the upper airway behaves like a collapsible tube. During normal conditions (open airway) the pressure in the pharynx (P_{PHARYNX}) is higher than the pressure exerted by the surrounding tissues (P_{TISSUE}). When P_{PHARYNX}>P_{TISSUE} the airway remains widely patent during breathing. (C) As P_{TISSUE} increases (e.g., sedation, decreased levels of conciseness), airway caliber decreases and airway resistance increases, resulting in a flow-induced drop in P_{PHARYNX}. This results in airway narrowing. (D) The moment when P_{TISSUE}>P_{PHARYNX}, the critical airway closing pressure is reached and inspiratory airflow stops.

FIG. 4A-4D. Prior art NPA versus the inventor's novel dilating and stenting NPA. (A), (C) Conceptual failure of a prior art NPA resulting in distal upper airway collapse. The multi-level nature of upper airway collapse can still cause airway collapse despite proper placement of a prior art NPA. The distal aspect of the inventors nNPA will self-expand ((B) lattice configuration or (D) fins configuration) when it is deployed into the pharynx and have larger resting diameter than the body of the nNPA, overcoming P_{TISSUE} and relieving airway collapse directly or by transmitting the airway diameter increase to surrounding tissues. Fenestrations or channels in the dilated segment will further augment airflow both nasally and orally and will allow for the oral evacuation of secretions from the pharynx and central lumen of the nNPA.

FIG. 5A-5B. Illustration of examples of a dilating and stenting nNPA in place (A) lattice configuration and (B) fins configuration.

FIG. 6A-6B. An illustration of an example of the distal aspects of nNPAs with (A) a lattice configuration and (B) a fin configuration.

FIG. 7A-7B. An illustration of one example of an nNPA having a fin configuration, (A) front perspective and (B) a side perspective.

FIG. 8A-8B. A perspective view and a front view of one example of the fin configuration nNPA, (A) a side perspective and (B) a front perspective.

FIG. 9A-9B. An illustration of one example of a NPA having a lattice distal configuration, (A) front perspective and (B) a side perspective.

FIG. 10A-10B. A perspective view and a front view of one example of the lattice configuration NPA, (A) a side perspective and (B) a front perspective.

FIG. 11A-11C. Illustration (full side view) of an example and mechanism of operation of an nlNPA that incorporates a central introducer—(A) shows an introducer in place and lattice collapsed, (B) cross-section showing an introducer in place and lattice collapsed, and (C) shows an introducer being removed and lattice expanded.

FIG. 12. Illustration (distal end side view) of an example and mechanism of operation of an nlNPA that incorporates a central introducer.

FIG. 13. Illustration (proximal end side view) of an example and mechanism of operation of an nlNPA that incorporates a central introducer, (A) cross-section of the assembly and (B) side view of the proximal end of the introducer.

FIG. 14A-14B. illustration, (A) perspective and (B) side view, of one example of an OA airway.

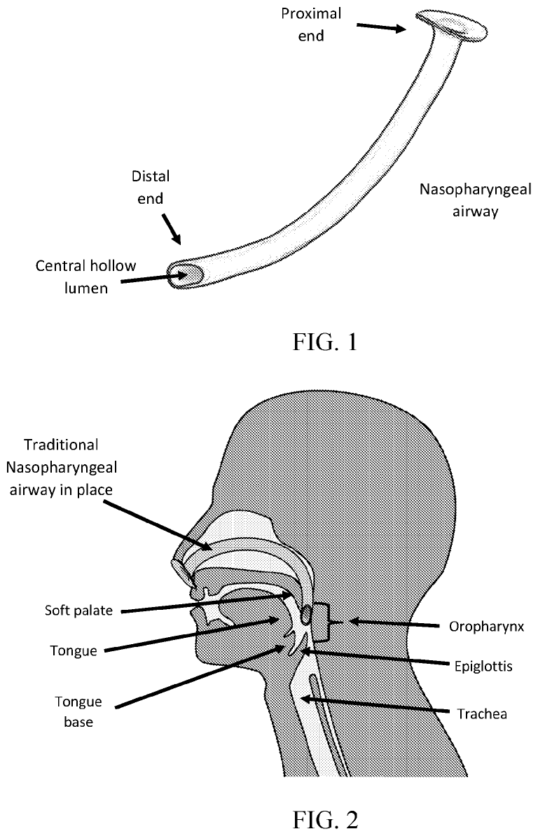

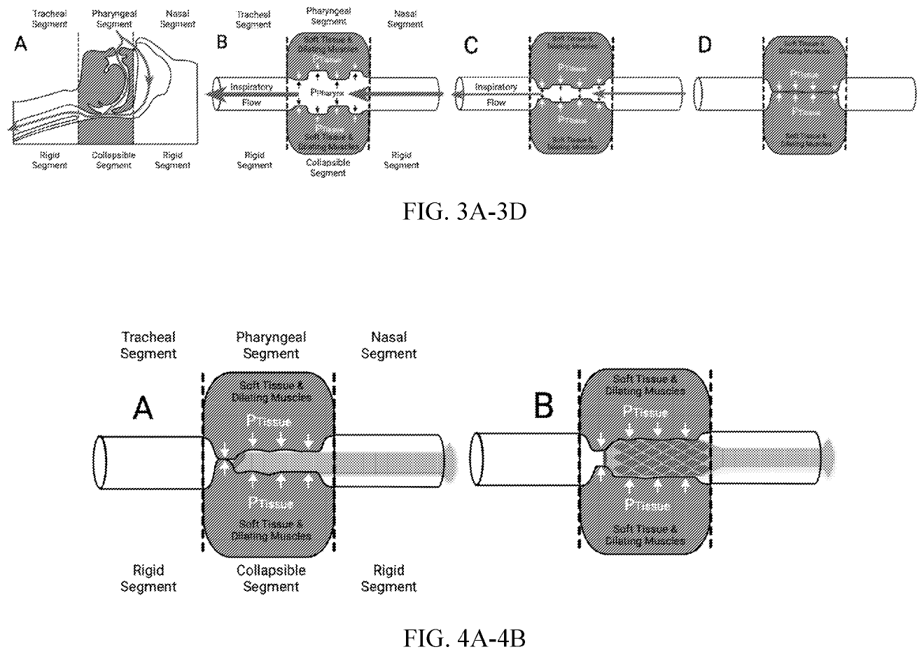

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

An NPA is placed in unconscious or semiconscious patients to maintain or restore upper airway patency and breathing during spontaneous ventilation or mask-assisted positive pressure ventilation. Despite the wide distribution of the NPA, which are found within virtually every emergency cart and airway kit, they are not universally effective in reliving upper airway obstruction and they appear to be underutilized.

The mechanisms and location of upper airway patency in patients with diminished levels of consciousness is much more complex than was recognized 140 years ago when the prior art technology was conceived. Upper airway patency depends on an appropriate balance between the structural components, the dilating force of the pharyngeal muscles, and the collapsing force of negative intraluminal pressure caused by inspiratory airflow. The neuromuscular activity of upper airway stabilizing muscles are greatly diminished during sleep, sedation, anesthesia, and altered states of consciousness such as the lethargy and coma that can be associated with shock, brain injury, and severe metabolic disturbances. These states are associated with decreased pharyngeal caliber, increased upper airway resistance, and ultimately upper airway collapse. The principal points of obstruction in the pharynx occur at the level of the soft palate, tongue base, central oropharynx and the epiglottis (FIG. 2). A properly sized standard NPA will pass below the soft palate with the tip residing approximately 1 cm above the epiglottis. This position will provide relief of obstruction at the soft palate level only if the distal tip or the lumen of the NPA does not become obstructed with secretions or soft tissue. However, the effectiveness of the NPA in reliably relieving obstruction at the level of tongue base, oropharynx, and epiglottis is not fully understood. In fact, despite the presence of an NPA, airway obstruction can occur in up to 42% of heavily sedated patients. This is not surprising given the contemporary understanding about the multi-level complexity of sedation-induced airway collapse through the advent of drug-induced sleep endoscopy (DISE) and drug-induced sleep computed tomography (DIS-CT) studies.

Orally inserted extraglottic airways (EGA) have emerged as an alternative to the NPA to treat upper airway obstruction. However, these larger EGA devices are limited to patients who are deeply unconscious, lack a gag reflex, and whose mouths can be opened enough to insert the large device. Insertion of an oral EGA in a semiconscious patient may not be tolerated and could cause gagging, vomiting, and aspiration. Furthermore, compared to an NPA the insertion of EGA's such as the i-gel® require more training, more knowledge, and much more skill to properly seat and confirm proper placement. EGA's also take up more space on packs, are heavier, and are more expensive than NPA's. By comparison, the NPA is easy to teach, easy to insert, and is better tolerated even in semiconscious patients. This makes the NPA a more attractive prehospital tool when the goal is temporary upper airway patency during spontaneous or mask-assisted breathing.

Although the simplicity and ease of use of the NPA is ideally suited for the relief of airway obstruction in hospital and prehospital environments, this 140-year-old technology does not fully address the complexity of upper airway obstruction and needs an innovative overhaul. The inventors designed an NPA that is effective in stenting open the entire upper airway, is easy to insert, minimizes airway trauma, and will allow for oral ventilation and the evacuation of secretions.

The improved NPA described herein can be effective in both prehospital and inpatient settings. The improved NPA described herein is easy to rapidly insert by even low-skill providers and will be more reliably relieve upper airway obstruction during spontaneous breathing and mask-assisted positive pressure ventilation. The NPA described herein expands the upper airway to alleviate obstruction and this luminal expansion is transmitted proximally and distally from the self-expanding section resulting in multi-level airway opening. Fenestrations or channels within the dilated segment will further augment airflow both nasally and orally and will allow for the oral evacuation of secretions from the pharynx and central lumen of the nNPA.

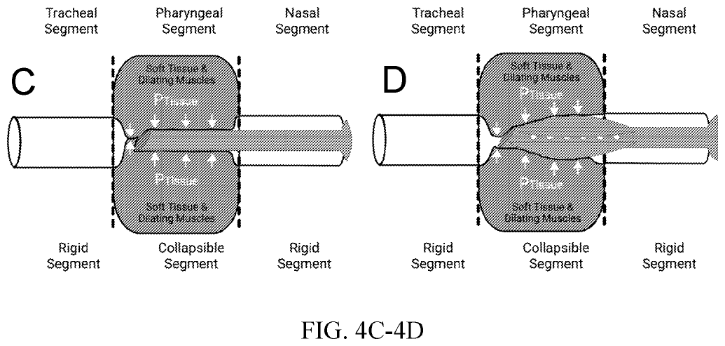

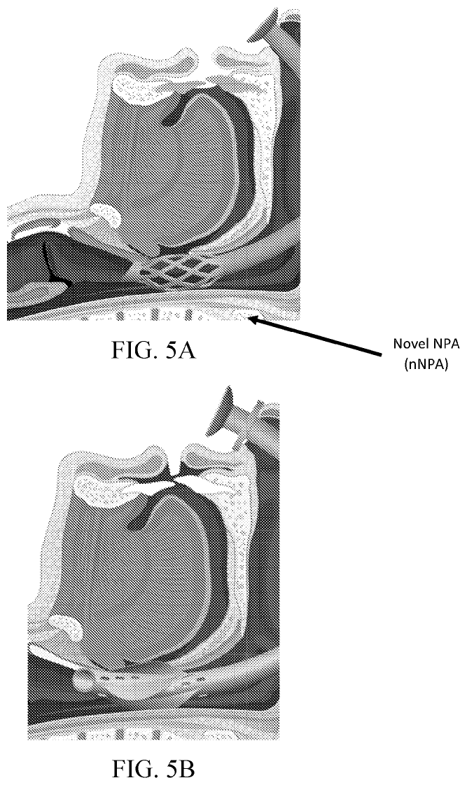

It is now known that obstruction can occur at multiple pharyngeal levels, and that the forces required to overcome collapse are quite negligible. There are currently no NPA devices available to address these challenges. FIG. 4A illustrates the conceptual failure of a standard NPA resulting in distal upper airway collapse. Unless the standard NPA tip fortuitously ends within an open pharyngeal segment and the airway distal to its tip is unobstructed, it is vulnerable to failure. FIG. 4B and FIG. 5 illustrate concepts of the nNPA described herein in that it has a larger distal resting diameter, overcoming P_{TISSUE} and will lift adjacent tissues, extending the dialating effect beyond the stenting section. Fenestrations or channels augment allow nasal and oral ventilation, allow flow in and out of the central channel, and allow for the evacuation of secretions.

Various engineering inputs have been established for the development of nNPA prototypes. Information such as desired performance, required lengths, length adjustability, diameters, Shore hardness, materials, biocompatibility, airway distending pressure, stenting matrix materials, fenestration size and shape, tip configuration, frictional coefficient, and other relevant inputs are determined to precisely guide design and engineering. Engineering inputs are also based upon an analysis of standard NPAs, anthropometric data, anatomic analysis, data obtained from existing sleep medicine research, information obtained from Drug Induced Sleep Endoscopy and Drug Induced Computed Tomography studies, as well as industry standards that pertain medical devices. Collectively, these inputs are used for innovation and nNPA design.

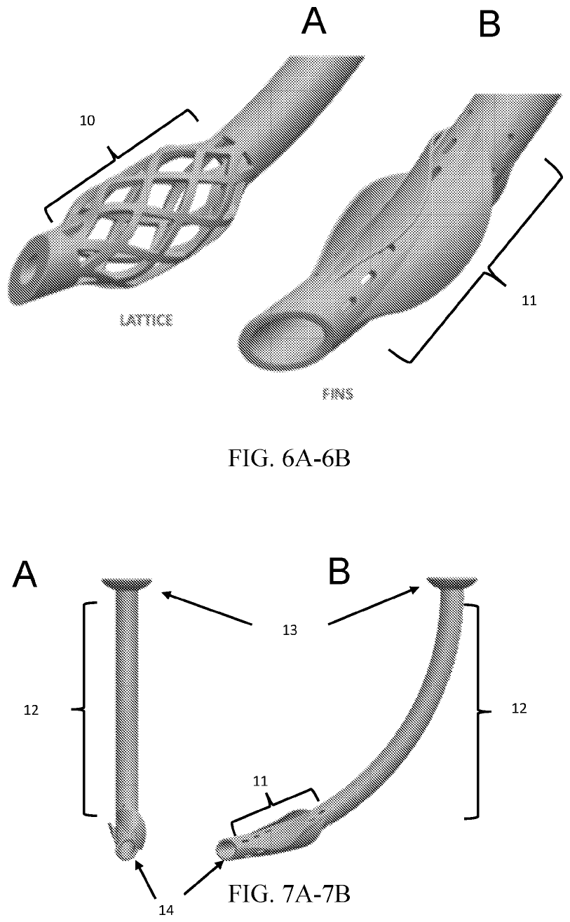

FIG. 6 illustrates examples of the distal aspects of nNPAs with a lattice configuration 10, and a fin configuration 11. The purpose of the lattice or fins is to stent open pharyngeal soft tissues in order to relieve airway obstruction where they contact tissue and to transmit airway expansion proximally and distally.

FIG. 7 illustrates a nlNPA having a fin configuration 11. Each nasopharyngeal airway (NPA) embodiment comprises an elongated body 12 forming a lumen, the elongated body having a proximal end 13 to prevent proximal NPA advancement past the nasal opening and will be positioned outside of a patient during use and a distal portion 14 to be inserted into a patient and positioned within the pharynx during use. The proximal end can be configured with a collar or other stop mechanism. The NPA is configured to be inserted in an airway with proximal portion 13 remaining outside of the airway. The distal portion has a functional configuration including a lattice or fins. The distal lattice 10 or fin 11 portions are configured to provide an expansion force to open an airway.

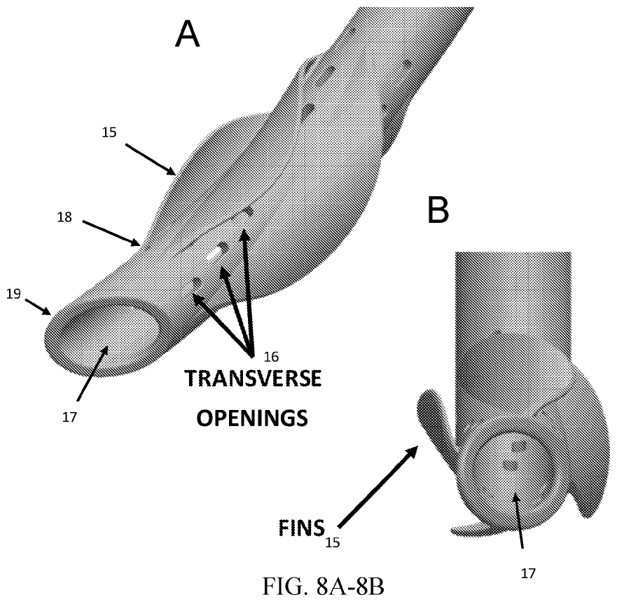

FIG. 8 is a perspective view and a front view of the fin configuration nNPA. The distal portion of illustrated in FIG. 8 has flat projections or individual fins 15 about the circumference on the exterior surface with the attachment of the fins 15 running at an angle to the long axis of the distal portion. The angle of the long axis of the fin with respect to the long axis of the distal portion can 1, 5, 10, 15, 20, 25, 30, 35, 40, to 45 degrees. The fins 15 can be semicircular or semi ovoid in shape and are configured to have curved surface that projects above the exterior surface of the NPA when in an open configuration. The fins 15 are flexible and can be folded flat without additional tools to ease insertion through nostril and nasopharynx and also provide an expansion force to open the more distal upper airway due to the propensity of fin material to return to its open state. The purpose of the fins is to stent open pharyngeal soft tissue to relieve airway obstruction where they contact tissue and to transmit airway expansion proximally and distally. The fins 15 can be 1, 2, 3, 4, 5, 6, 7, 8, 9, 10, 11, or 12 cm in length or any fraction therein. The fins 15 can be part of a single-piece part (as shown) or attached after the NPA body is formed during manufacture. The space between the fins 15 may include transverse openings or fenestrations 16 that pass through the wall of the distal portion of the nNPA and fluidly couple the interior lumen of the nNPA 17 to the space exterior to the device. The transverse openings, channels, or fenestrations serve to augment airflow both nasally and orally and will allow for the oral evacuation of secretions from the pharynx and central lumen of the nNPA when the nNPA is deployed in a subject. The transverse openings, channels, or fenestrations may consist of individual openings between each fin segment or may be singular and span the entire length of each fin segment. The distance between the distal edge of the fins 18 and the distal end of the NPA 19 can 0, 1, 2, 3, 4 cm or any fraction therein. The number of fins 15 on the device can be 2, 3, 4, 5, 6, 7, or 8.

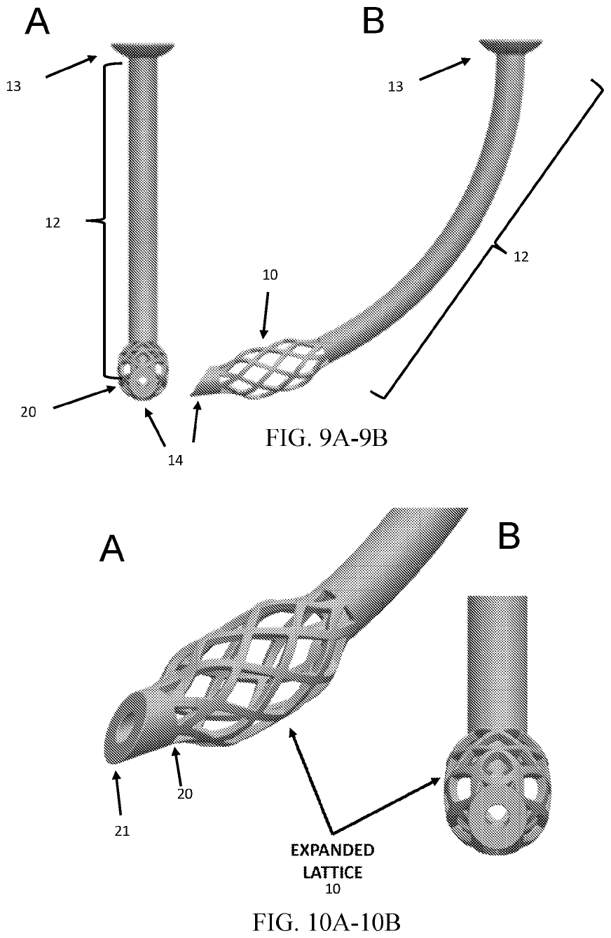

FIG. 9 illustrates a NPA having a lattice distal configuration. Each nasopharyngeal airway (NPA) embodiment comprises an elongated body 12 forming a lumen, the elongated body having a proximal end 13 to prevent proximal NPA advancement past the nasal opening and will be positioned outside of a patient during use and a distal portion 14 to be inserted into a patient and positioned within the pharynx during use. The NPA is configured to be inserted in an airway with proximal portion remaining outside of the airway. The distal portion has a functional configuration including a lattice or fins. The distal lattice or fin portions 10 and 11 are configured to provide an expansion force to open an airway. The lattice portion 10 has an expandable structure and action that is configured to be positioned in the pharyngeal segment of a patient airway. The self-expanding lattice structure is collapsible and can be collapsed to a smaller diameter without additional tools to ease insertion through a nostril and the nasopharynx. The lattice portion 10 also provide a self-expansion force to open within the more distal upper airway due to the propensity of the integral properties of the material, or an internal structure, to return to its open state. The purpose of the lattice is to stent open pharyngeal soft tissue to relieve airway obstruction where it contacts tissue and to transmit said airway expansion proximally and distally. The purpose of the openings or fenestrations within the open lattice are to augment airflow both nasally and orally and allow for the oral evacuation of secretions from the pharynx and central lumen of the nNPA when the nNPA is deployed in a subject.

FIG. 10 is a perspective view and a front view of the lattice configuration nlNPA 10. The distal portion illustrated in FIG. 10 has an expandable lattice 10 about the circumference of the distal portion of the nl NPA. The lattice having interconnected strands attached at points along the lattice forming openings in the body wall. Each component of the lattice 10 forming an angle with respect to another lattice component at the point of contact, the angle 1, 5, 10, 15, 20, 25, 30, 35, 40, 45, 50, 60, 65, 70, 75, 80, 85, to 90 degrees. The lattice 10 is configured to have an outer diameter that is larger than the exterior surface of the NPA body 12 which can be 0×, 1×, 2×, and 3× the width of the nlNPA body and all fractions therein when in an open configuration. The length of the lattice structure can be 1, 2, 3, 4, 5, 6, 7, 8, 9, 10, 11, or 12 cm in length or any fraction therein. The lattice portion is flexible and can be reduced in diameter without additional tools to ease insertion through nostril and can open when in the pharynx to provide an expansion force to open airway due to the propensity of lattice material to return to its open state. The openings in the lattice 20 augment airflow both nasally and orally and will allow for the oral evacuation of secretions from the pharynx and central lumen of the nNPA when the nNPA is deployed in a subject. The lattice 10 can also be contracted using an introducer to ease insertion/removal by maintaining the lattice 10 in a contracted state until deployed in the airway. The configuration of the lattice 10 provides an expansion force to open airway when introducer is removed. Lattice 10 may be integrated into the part (as shown), may be attached, and may have an internal reinforcing and/or expanding structure. The distance between the distal edge of the lattice structure 20 and the distal end of the NPA 21 can 0, 1, 2, 3, or 4 cm or any value or range there between.

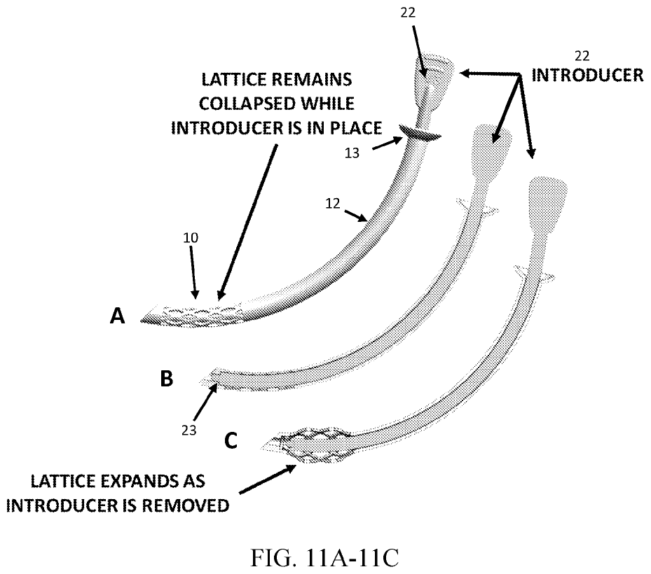

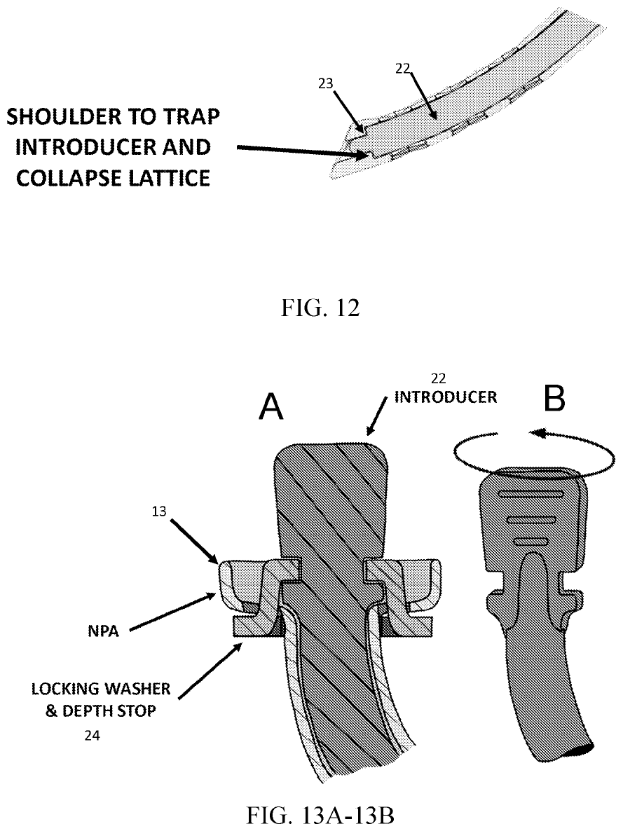

FIG. 11 to FIG. 13 illustrate an example and mechanism of operation of an nlNPA that incorporates a central introducer. An introducer can facilitate nlNPA insertion, and/or maintain the lattice in a collapsed state prior to and during nlNPA placement, and/or permit lattice expansion once the introducer is removed. FIG. 11A shows the introducer 22 in the pre-insertion position. Introducer 22 is configured to be inserted into the central lumen of the nlNPA through the proximal opening of the nlNPA. FIG. 11B and FIG. 12 are cross sections of the nlNPA with an introducer that demonstrates that when the introducer 22 is fully inserted it reaches a shoulder 23 at the distal portion of the nlNPA inducing an axial load on the distal portion of the lattice causing it to collapse and decrease in diameter. Introducer 22 decreases the diameter of the lattice and provides stability of the lattice 10 to ensure that it can be inserted into the patent's airway without collapsing. In certain aspects introducer 22 can be inserted after NPA use to collapse lattice 10 prior to removal. FIG. 11A and 11B shows aspects where the lattice 10 remains collapsed while introducer 22 is in place. FIG. 11C shows that the lattice 10 expands under its own force as the introducer 22 is being removed. In certain aspects the internal surface of the NPA body 12 is structurally configured to receive introducer 22 by having a shoulder 23 to trap and collapse lattice 20 (see FIG. 12).

FIG. 13 illustrates a locking mechanism for introducer 22. Locking washer 24 captures introducer 22 in place, ensuring introducer stays in place and lattice 10—remains collapsed. Introducer 22 is twisting to unlock, then removed. After removal of introducer 22, washer 24 functions as depth stop to adjust NPA placement depth inside airway.

Oral airways, like their NPA cousins, were introduced into the practice of anesthesia and cardiopulmonary resuscitation several decades ago to also provide upper airway patency during anesthesia, deep sedation, or obtundation from other causes. The oral airway is defined by the mouth, the oral cavity, the tongue, and the epiglottis. An oral airway device OA is positioned within the patient's oral airway, with the distal end providing structure to prevent the collapse of soft tissue structures, and the central portion providing a conduit for airflow.

Certain embodiments are directed to an oral airway (OA) comprising an elongated body or body member forming a lumen, the elongated body having a proximal end to be positioned outside of a patient during use and a distal end to be inserted into a patient's oral airway during use.

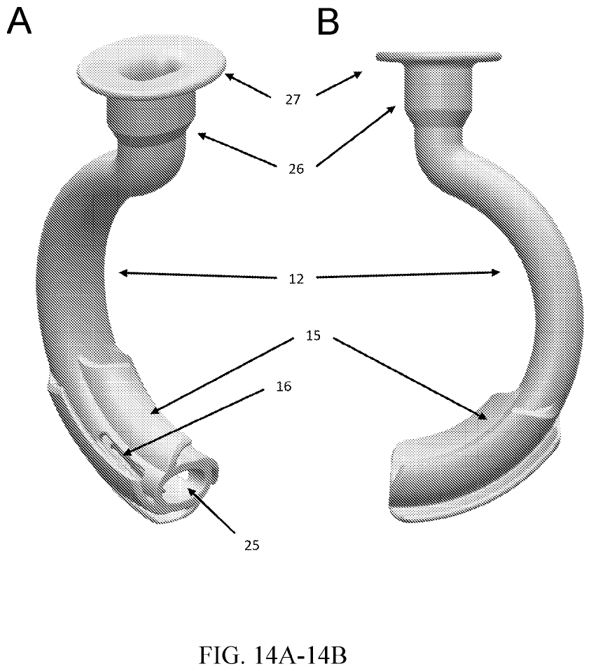

FIG. 14 illustrates an OA version of the airway opening technology detailed herein. The elongated body 12 having a distal expandable portion or segment configured to be positioned in the oral airway of a patient or subject which when expanded or self-expanded provides airflow patency within the patient's upper airway in which the OA is inserted due to stenting open of the soft tissues or preventing collapse of soft tissues. The distal expandable portion can be either a lattice or fins 15 configuration. The airflow patency is also provided or enhanced using the central lumen 25 of the OA, and through transverse openings or fenestrations 16, and/or fins 15. In certain aspects the expandable portion includes foldable fins 15 and transverse openings or fenestrations 16 forming a fluid path between the inner lumen and exterior of the OA. The expandable portion can include a collapsible and/or self-expanding lattice structure with fenestrations forming a fluid path between the inner lumen and exterior of the OA. The OA can be 3, 4, 5, 6, 7, 8, 9, 10, 11, 12, 13, 14, 15, 16, 17, 18, 19, or 20 cm in length (including all values and ranges there between), with an outer diameter ranging from 0.5, 1, 2, 3, 4, 5 cm, and an inner diameter of 0.1, 0.2, 0.3, 0.4, 0.5, 0.6, 0.7, 0.8, 0.9, 1, 1.5, 2, 2.5, 3, 3.5, to 4 cm (including all values and ranges there between). The distal expandable portion is 0.1, 1, 2, 3, 4, to 5, 6, 7, 8, 9, to 10 cm in length (including all values and ranges there between) and having an expanded outer diameter of 1, 1.5, 2, 2.5, 3, 3.5, 4, 4.5, 5, 5.5, to 6 cm (including all values and ranges there between). The distal expandable portion can be positioned 0.1, 0.25, 0.5, 1, 2, 3, to 4 cm (including all values and ranges there between) from the distal tip or the beginning of a distal beveled tip of the body. The OA can be made of a polymer material. The distal most 0.2 to 3 cm (including all values and ranges there between) can be a flat or beveled tip. The OA can be coupled with a central introducer to facilitate insertion of the OA into a patient which can, but not necessarily provide one or more of axial rigidity, collapse a self-expanding lattice structure, and/or allow the lattice structure to expand as the introducer is removed. The central introducer can be temporarily secured in place by a locking mechanism on the proximal end of the OA. The OA can be a curved hollow, tubular longitudinally extending body. In certain aspects the curvature of the body is between 0° 10, 20, 30, 40, 50, 60, 70, 80, 90, 100, 110, 120, 130, to 140°. The body can include a flanged proximal end portion for location at the mouth of the patient. In certain aspects the flanged proximal end portion is configured as a bite block or is configured to connect to a bite block. The body member further includes a distal end portion for insertion into the mouth and pharynx of the patient.

The body or distal portion (e.g., the distal 10, 20, 30, 40, 50, 60, 70, 80, 90, 91, 92, 93, 94, 95, 96, 97, 98, or 99% of the body length, including all values and ranges there between) of the body of the OA can be of sufficient softness and pliability to bend during insertion and to accommodate different angles once it is successfully inserted into the patient, since a given patient's head and neck may be slightly flexed or extended. In certain aspects the most proximal end portion (e.g., the proximal 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% of the body length, including all values and ranges there between) of the OA is harder than the distal end portion in order to prevent occlusion by the patient biting down thereon.

At the proximal end of the body can be a bite block 26 having a flanged end 27. The bite block can be integrally formed from a thickened side of the tubular member or be a more rigid plastic collar that is slid over or connected to the proximal end and held in place by friction, an adhesive, mechanical coupling (e.g., threads, latch, snap, etc.), or overmolding. In addition, an adaptor may be placed inside the flanged end of the bite block and may be permanently affixed or releasably held in place by means of friction or other mechanisms. It may also be integrally formed with the bite block in a single manufacturing step.

1. An airway device comprising an elongated body forming a lumen, the elongated body having a proximal end to be positioned outside of a patient during use and a distal end to be inserted into a patient airway during use, the elongated body having a distal expandable portion configured to be positioned in the pharyngeal segment of a patient airway which when the distal expandable portion expands in the pharynx a structure is deployed stenting open of soft tissues increasing the luminal diameter of the airway and providing an alternate path for airflow, the alternate path for airflow comprising the airway lumen and/or fluid paths provided by fin structures, lattice structures, or fenestrations.

2. The airway device of claim 1, wherein the expandable portion comprises foldable fins and transverse openings or fenestrations forming a fluid path between the inner lumen and exterior of the airway device.

3. The airway device of claim 1, wherein the expandable portion comprises a collapsible and self-expanding lattice structure with fenestrations forming a fluid path between the inner lumen and exterior of the airway device.

4. The airway device of claim 1, wherein the airway device is 8 cm to 30 cm in length, with an non-expanded outer diameter ranging from 0.4 cm to 5 cm, and a non-expanded inner diameter of 0.2 cm to 3 cm.

5. The airway device of claim 1, wherein the distal expandable portion is 2 cm to 10 cm in length and an expanded outer diameter of 1 cm to 6 cm.

6. The airway device of claim 1, wherein the airway device is made of a polymer material.

7. The airway device of claim 1, wherein the distal most 0.2 cm to 5 cm is a beveled tip.

8. The airway device of claim 1, further comprising a central introducer.

9. The airway device of claim 8, wherein the central introducer is axially rigid and is configured to retain the self-expanding structure in a collapsed state until deployed.

10. The airway device of claim 8, wherein the central introducer is temporarily secured with a locking mechanism on the proximal end of the airway device.

11. A method for inserting the airway device of claim 1 comprising of advancing a distal end of an airway device through a nostril opening or an oral cavity into a patient airway and caudally into the pharynx, the airway device having an expandable proximal end in a collapsed configuration during insertion, wherein the collapsed configuration is reversibly retained by an introducer, mesh, filament, cord, or string;
expanding the expandable proximal end in the pharynx or releasing the retention of the collapsed state thereby deploying stenting fins or a lattice structure increasing the luminal diameter of the airway device and providing alternate paths for airflow.

12. An airway device comprising an elongated body forming a lumen, the elongated body having a proximal end to be positioned outside of a patient during use and a distal end to be inserted into a patient airway during use, the elongated body having a distal expandable portion configured to be positioned in the pharyngeal segment of the patient airway which when the distal expandable portion is expanded in the pharynx a structure is deployed stenting open of soft tissues increasing the luminal diameter of the airway and providing an alternate path for airflow, the alternate path for airflow comprising the airway device lumen and/or fluid paths provided by fin structures, lattice structures, or fenestrations.

13. The airway device of claim 12, wherein the airway device is configured as a nasopharyngeal airway (NPA).

14. The airway device of claim 12, wherein the airway device is configured as an oral airway (OA).
